(19) United States
(12) Patent Application Publication
(10) Pub. No.: US 2011/0129577 A1
(43) Pub. Date: Jun. 2, 2011

(54) PROCESS AND MACHINE EQUIPMENT FOR THE CONTROL OF QUALITY OF BAKERY PRODUCTS

(75) Inventors: Benjamin DE CHILLY, La Bastide L'Eveque (FR); Emmanuel DUMAS, Villefranche De Rouergue (FR); Thierry DE LAZZARI, Honor De Cos (FR); Christian MADAULE, Jonquieres (FR)

(73) Assignee: DE LA BALLINA FRERES, Maleville (FR)

(21) Appl. No.: 12/893,596

(22) Filed: Sep. 29, 2010

(30) Foreign Application Priority Data
Sep. 30, 2009 (FR) 09/04673

(51) Int. Cl. G01N 33/10; A23L 1/01; A47J 37/00

(52) U.S. Cl. 426/233; 99/325

(57) ABSTRACT

In an industrial bakery system, the control of quality of bakery products is carried out by a process comprising submitting said products when present in their respective locations on a moulding plate to a first visiometric inspection step wherein they are inspected from above and digital image data are produced comprising at least location data indicative of their respective positions on said plate, then seizing individually said products out of said plate depending on said location data by prehensors arranged in a gripping matrix of prehensors, and further submitting said seized products to a second visiometric inspection step wherein they are inspected from underneath while they are hanging from said gripping matrix.

This invention concerns the control of products as part of an industrial production line. It is especially advantageous for controlling bakery products that have gone through the baking stage under real conditions, in particular in the domain of industrial bakery, when one conducts a quality control process using visiometric means before excluding from packaging circuit products which do not meet the predetermined shape or aspect specifications.

It is within the context of such application, which however does not have a limitative effect, that the Applicants have already described the technology in various forms, in the specifications of patents and patent applications that belong to the Assignee.

In those patent descriptions as in the present case, “visiometry” means the technique used to acquire digital images of products to be examined under the light of a laser beam, and to submit the acquired images to a digital image signal treatment process for the automatic calculation of the shape and aspect information data in order to compare them to the predetermined quality thresholds and for the automatic generation of sorting command signals according to the comparison results for each product. In order to clearly understand the present description if necessary, one will be able to, in particular, refer to the published patent applications US 2005/099620, US 2009/245616, EP 2105217, EP 2105393 and WO 2010/067190.

In the texts referred to, one is especially interested in visiometric systems which are in place for the purpose of examining products that are to be led continuously via the visiometric device, in the field of vision of one or more cameras for acquiring digital images. The images are acquired in a repetitive manner during the relative displacement between each product and the lit field of vision, whether it is linear cameras or matrix type cameras that are used. The systems described in the patents and patent applications above mentioned are specially recommended for the treatment of products which are deposited in bulk on the surface of the conveyor belt via the visiometric device to extract them according to information about quality and counting, elaborated via digital treatment of the obtained images.

In the most current application cases, the technique is used with cooked or half-cooked products in a frozen state, taken from a frozen environment and either led to different reception plates depending on whether or not they conform to the quality demands or separated in successive lots which each contain a predetermined number of products which have been judged as conforming to such demands with regard to their delivery packaging.

The current invention has its main objects in a different context, involving applying the visiometric analysis technique principle of which has just been recalled during the quality control of the products that arrive correctly aligned at the visiometric inspection step.

An object of the invention relates to a quality control process in an industrial bakery installation, wherein bakery products that are present in their respective locations on a moulding plate are submitted to a first visiometric inspection step wherein they are inspected from above for producing digital image data including at least an information about their location on said plate and wherein said products are then submitted to a second visiometric inspection step wherein they are inspected from underneath while they are hanging from a matrix of prehensors by which they have been seized and lifted out of the corresponding moulding plate.

According to another aspect of the invention, means are advantageously provided to ensure, via an automatic driving system, the control of the different prehensors in the gripping matrix for governing the release of the products that they have seized, according to information about location, presence and quality, which are obtained by data processing from the digital images acquired during the visiometric inspection steps, so as to direct the different products to different circuits out depending on whether or not they conform to predetermined quality specifications.

The visiometric inspection step from above can further be carried out so as to check the flatness of the plates, in order to determine whether or not each plate can be re-used for a new moulding operation. Such verification the plate shape has been preserved is especially useful when the moulding plates are cooking plates used to carry the products moulded in mould cavities of the plate through an oven. The information is then used to decide whether each plate can be recirculated to the oven for receiving a new batch of products.

The invention also concerns a quality control equipment with all appropriate means for the implementation of the process.

The various characteristic features of the present invention are detailed hereinafter, sometimes in different variants, with the understanding that the detailed description of the variants is not limitative with regard to the implementation forms that the process and the installation can take on within the framework of the invention.

An installation constructed in accordance with the invention specifications is illustrated by the figures, among which figures:

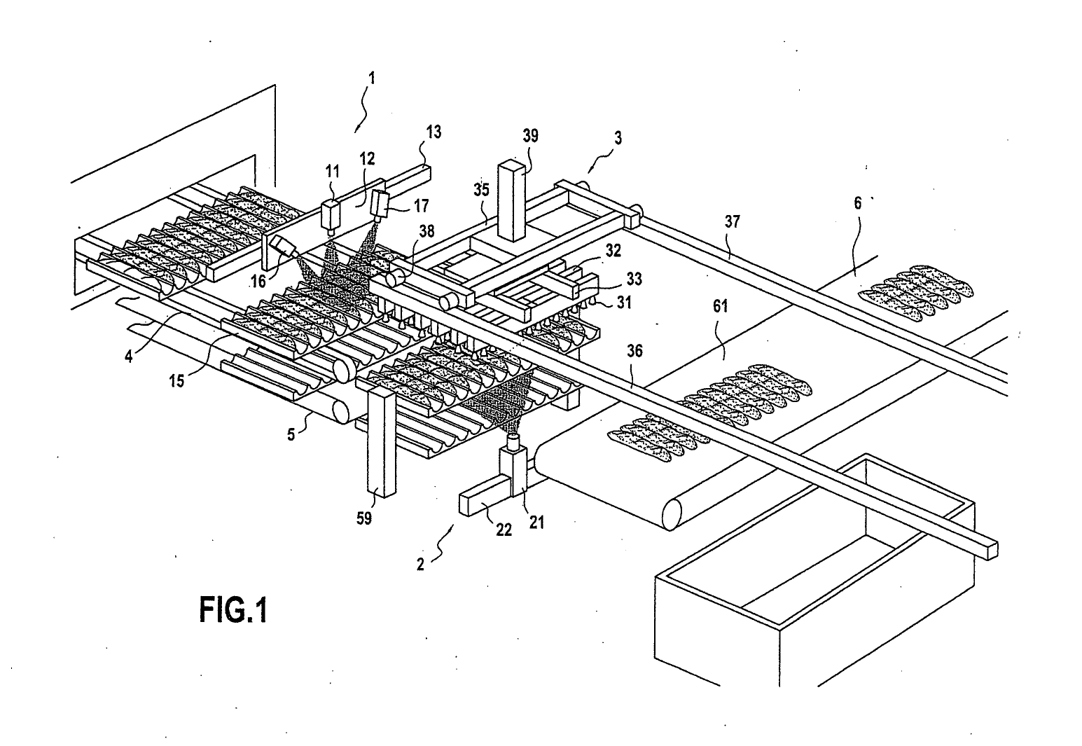

FIG. 1 diagrammatically illustrates the different essential elements of the installation in a perspective view,

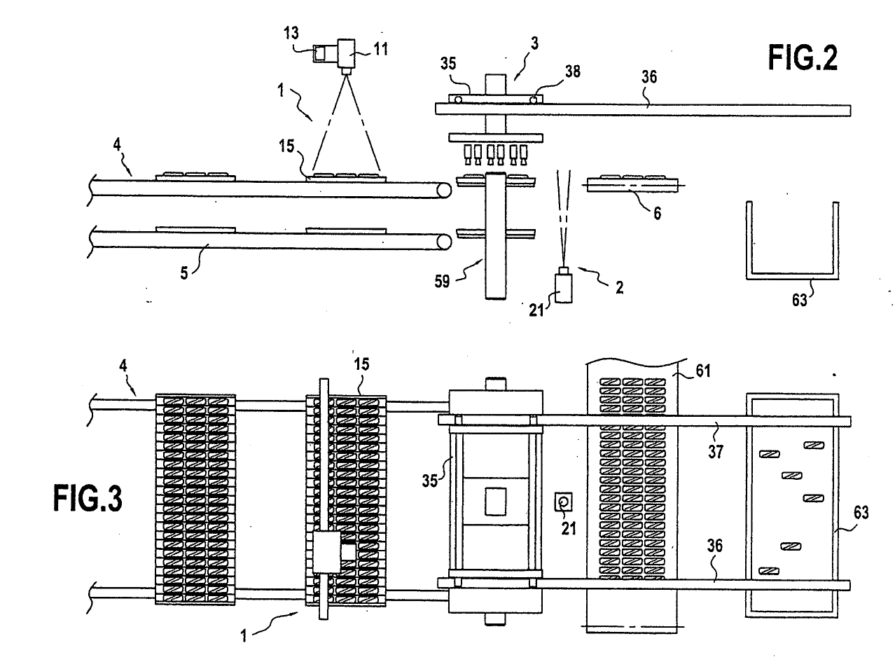

FIG. 2 diagrammatically illustrates the same installation, viewed from above,

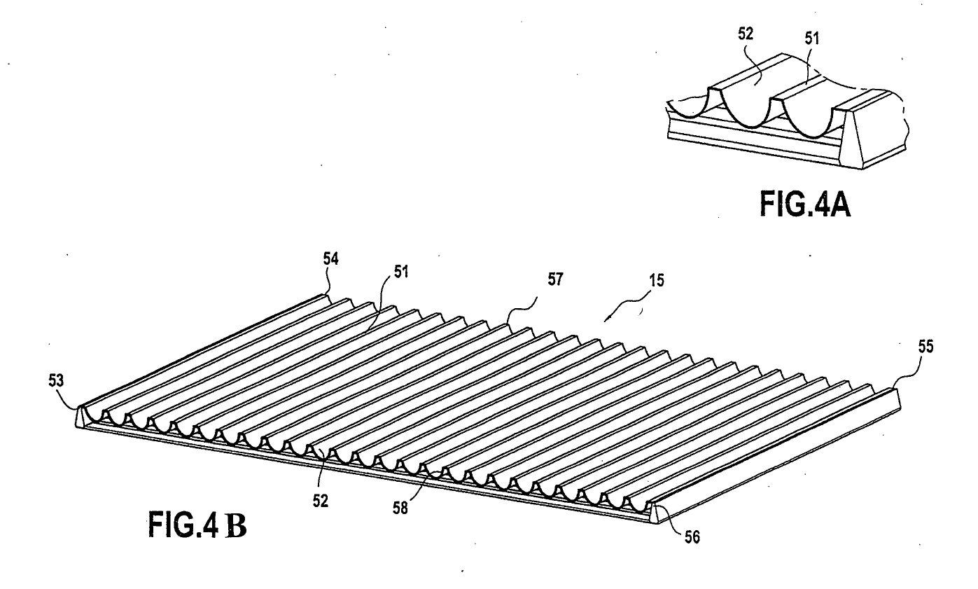

while FIG. 3, in parallel, illustrates it in plan view,

FIG. 4 represents a cooking plate with cavities receiving elongated forms of bread (baguette-type),

FIG. 4A shows a detail of FIG. 4 which highlights the presence of flat areas between cavities which are used to verify the flatness of the plates before reinserting them in the circuit for a new baking operation,

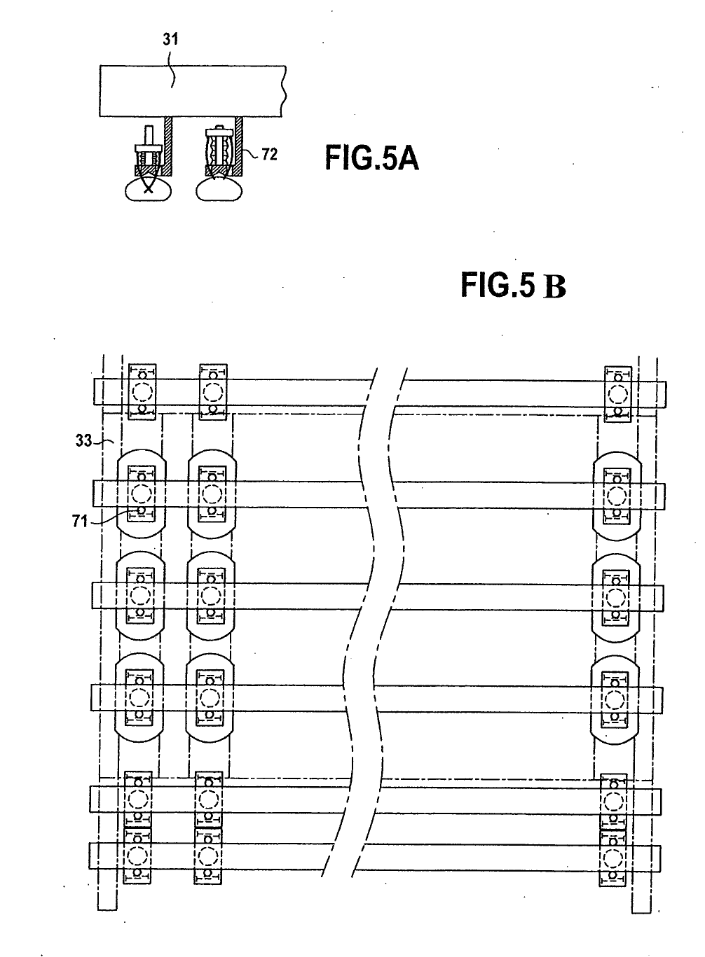

FIG. 5 diagrammatically illustrates, in plan view, the constitution of a gripping matrix, made of multiple prehensors for the transportation of suspended products,

FIG. 5A illustrates a part viewed from above, in the area of fixation of a pair of prehensors,

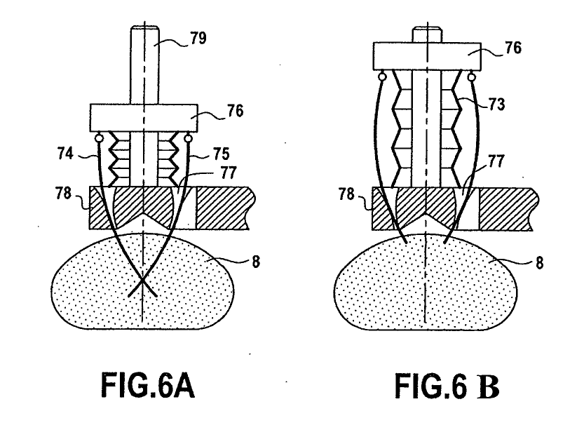

FIGS. 6A and 6B illustrate the functioning of one of the prehensors in the case of a needles prehensor system.

The installation illustrated by FIGS. 1, 2 and 3 is described in the framework of its application for quality control of bakery products that are cooked in moulds.

The visiometric inspection of products is effected just as products leaving the cooking oven are removed from cooking plates which are led outside of the cooking area by conveyors on which the plates are positioned in series, one after the other.

In this particular case, such a cooking plate is illustrated by FIG. 4. In this figure and in FIGS. 1 and 3, it may be observed that some cavities are managed hollowed in the entire width of the plate, to form reception areas for products. Here, the cavities are equidistant and parallel from each other in the direction of the conveyor transporting them from the cooking area. In practice, these plates are used for bread specimens that are baguette form, either in the form of traditional and complete French baguettes or in the form of baguette fractions as illustrated in FIG. 3, with one-thirds of the traditional baguette.

The visiometric inspection is conducted in two visiometric devices that operate one after the other, in the direction of the movement of plates containing products. The first visiometric device 1 examines baked products from above while they are still contained in the cooking plates. The second visiometric device, indicating the reference 2 on the figures, examines the products themselves from underneath when they have just been extracted from their respective location of the moulding plate and when they are hanging from a transport system 3.

This transport system, as indicated in the implementation modes of FIGS. 1 to 3, is supposed to be composed of a matrix of suction prehensors', such as that which has been described in the patent applications of the Depositor EP 2105217 and EP 2105393. There is a description there not only of the mechanical constitution of the individual prehensors and their regrouping in lines and columns within the matrix, but also of the IT system for the treatment of data which automatically creates the individual prehensor commands based on information registered via acquisition of the digital images taken in an upstream visiometric device. It also contains an ample description of the transport system for the suspended products via suction prehensors in the event that it is used downstream of a visiometric device which examines the products from above to make them pass, in a suspended state, via a visiometric device which examines the products from underneath. The images acquisition operations were realised while the products were led underneath a fixed set of laser lighting and camera(s).

The visiometric device 1 is carried out with products that are still present in their respective locations on the cooking plate, while said plate is in a fixed, position on the entrance conveyor 4. In the particular implementation mode described, there is therefore the preference to use a set for obtaining scanning images. This is how FIG. 1 diagrammatically illustrates the fact that the laser camera is installed on a movable truck 12 which is led, by non illustrated driving means, to move along a beam 13, which guides the said truck in a translation transversally to the conveyor that transports the plates. During the operation, the images are thus acquired product by product, the baguettes being observed one after the other, during the displacement of the camera above the immobile plate, from one side of the conveyor to the other.

FIG. 1 still illustrates that in the camera equipment, laser lighting 11 emits a planar bean and that, on the plate 15 being treated and the products that it contains, the laser light plane extends over the whole width of the plate, in the longitudinal sense of the conveyor.

In addition, the particular realisation of an implementation following the invention as illustrated by the figures, is appropriate for taking three-dimensional (3D) images. This is why the visiometric inspection camera equipment is similar to what has been described in a previous patent application for the Depositor US 2009/245616, with two cameras for acquiring images 16 and 17 functioning in co-operation with the same laser lighting source 11 and directed toward the trail of light by laser beam from both sides of the emission source. The exploitation of the digital images signals uses known per se means to supply information on height above a reference level which is connected to the usual conveyor plane, besides the location information for each image pixel in abscissa and ordinate in the products base plane.

The three-dimensional visiometry is here used for controlling the flatness of the cooking plates. It is known that the cooking plates can be reused for a substantial number of cooking operations for successive bread series, but that they become unusable when, due to the influence of heating and cooling phases, they get deformed, especially by taking flexion stress. In practice, every plate buckling creates a risk of having products that may be refused by customers for shape or cooking regularity matters.

The verification of the flatness of each plate comprises a step for determining height of the flat areas of the plate above the reference level. With image data treatment that involves the use of classic IT means, one can configure the system in order to detect the level of a flat 51 between two cavities 52 (see FIG. 4A) at different places of one plate, notably at its four angles 53, 54, 55 and 56 (see FIG. 4), and if necessary at other plate points, for example on its sides at the ends of the cavities, as in 57 and 58. The IT system calculations will allow one to deduce the degree of flexion stress that the plate may have suffered, for comparing the value obtained with a predetermined tolerance value, in order to decide automatically if the corresponding plate can be send back into the cooking oven or if it must be eliminated from the circuit.

It must be stated here that it is equally based on height information, this time focusing on the cooked products present on the cooking plate, that one can determine the own height of each bread specimen at each point. The depth of cavities, which form reception areas compared to the flat surfaces 51, is known from the construction data.

In case it fails to emerge in a sufficiently clear manner from the foregoing explanations, let us emphasise that the successive plates are led one after the other by the conveyor 4 to be fixed in the visiometric device thanks to an automatic stop of the conveyor when a new plate arrive as far as an appropriate abutment.

The installation is built in such a manner that it designates automatically the destination of each plate according to the result of the flatness verification made at the visiometric device 1. Every plate that is unusable is eliminated from the plates return circuit in the direction of a new cooking phase which, for the other plates, is ensured by a conveyor 5 located beneath the conveyor 4 parallel to it, with reverse-direction driving.

The transfer of the plates which are considered as correct is effected by a lift, diagrammatically illustrated by 59, which receives every full plate leaving the visiometric inspection device 1 and which maintains it at the high level of conveyor 4 during the time that is necessary for extracting products contained in the cavities by the transfer system 3, and which moves it down at the low level of the return conveyor 5 and make it pass on, seeing that it is about a plate that is not considered as defective. Naturally, all the corresponding commands are automatically synchronised with the other operations ensured by the installation.

As it has already been indicated, the extraction of products is effected by a collection of prehensors 31 that are, here, of the pneumatic type with suction cups. Each prehensor operates just like an automatically piloted suction device, to seize the product located underneath it and later liberate it via a jet of air.

In the particular embodiment illustrated by FIG. 1, as besides in that of FIG. 5, the prehensors 31 are distributed in series, each series having a linear arrangement transversally to the conveyor direction, with each series being located on a bar 32. The different bars 32 are made movable on two side members 33, which allows to adapt the number of prehensors on each column overhanging the bread specimens on their length according to the width of the plates or according to the number of requested prehensor points in their distribution on the length of the cavities.

The thus-developed gripping matrix is a part of the transfer system 3. With that in mind, it is mounted on a truck 35 that is movable in translation on the guiding rails 36 and 37, on which it is positioned by rollers 38. The truck displaying the gripping matrix is led by the intermediary of a support arm 39 by driving means (not illustrated) which are placed under the command of the automatic piloting system of the installation set.

For each loaded cooking plate that gets passed by the visiometric device 1, the different prehensors are controlled for the purpose of gripping all the products present in the cavities while the system 3 is maintained stationary above the plate retained at the high level of the lift 59. Let it be indicated that, given that the moulded cooked products being aligned in their respective cavities, the number of prehensors can be significantly reduced vis-à-vis the number that would be necessary to seize products which are in a random layout on a transport belt.

It is when products have to released that the prehensors are controlled on a varying basis according to results of comparisons effected for the purpose of distinguishing those products which do conform to the quality demands from those that do not. After the movement of the transfer system 3, products that are considered standard are released on exit conveyor 6. They are received on a transport belt 61, which drives them in a perpendicular direction to the main conveyor 4. When applied in usual industrial manufacturing operations, they are led to a freezer, to be frozen before being delivered. Products that are considered not in accordance with quality demands are led a bit further on the circuit of the rails 36 and 37, to be dropped in an evacuation container 63.

The products which are hanging from the transfer system 3 traverse the second visiometric inspection step before arriving in the position where they are to be released on the exit conveyor 6. They are examined from underneath by a laser camera 21 positioned on an installation crossmember 22 (on FIG. 1). At this step, it is generally preferred to use a simple linear camera, which will examine the different products in parallel as each product passes above its field of vision. Also at this step, surface information is generally enough for a correct analysis of whether or not the product is standard, insofar as information complements the information that has already been registered based on digital images acquired at the level of the first visiometric device 1.

In many practical application cases, it may be provided for checking conformity with regard to quality specifications concerning the presence, the shape, and the number of scarifications at the level of the visiometric from above device 1, while one is rather more interested in the presence of abnormal stains on the underside of the bread specimens at the level of the visiometric from underneath device 2. Furthermore, with the chosen example of implementation mode of the invention, it is advantageously provided to use image data which are issued from initial visiometric operations (visiometric from above, when products are still in the cooking plates before they are gripped and transferred to the installation exit point) for the purpose of deducing from them the location information which are used for driving the individual prehensors, while the image data of the second visiometric operations (visiometric from underneath device 2) are used for the purpose of checking that each product extracted from the plate is present in the transfer system. Various quality information can be deduced from the images obtained from one or other of the visiometric devices, notably in connection with the coloured intensity variations which indicate the degree of cooking, the density of grains decorating the upper surface of the bread specimens, the presence of stains due to rests of previous cooking operations that have been left at the bottom of the moulds, and cooking defects due to paste defects or to high stoking areas in the oven.

We will now look at the description of a variant of implementation of the installation which, in accordance with the invention, fulfill the own feature concerning the system for driving products from the visiometric from above device to the circuit out, to then pass above the visiometric from underneath device. This realization variant is specifically adapted to products which are still not yet hardened, neither by cooking nor by freezing. Therefore, it is, in particular, about bread specimens taken from cooking ovens in a cooked or half-cooked state, with crumb remains soft.

While suction-type prehensors are particularly adapted to rigid surface products, at least on their upper surface, as it is notably the case with products which have gone through a freezing phase while their alignment is maintained in the cavities of a plate similar to cooking plates, one prefers here in accordance with the invention to use needles prehensors when the products are supposed to be supple.

It can be observed that way at FIG. 5 a gripping matrix wherein the same bars 32 as those illustrated by FIG. 1 each carry a series of individual needles prehensors 71, each of which is constituted as it appears more clearly in FIGS. 6A and 6B. In the particular case illustrated, each prehensor is fixed underneath the corresponding bar 32 by a bracket 72. In other application cases it may be provided that their position be adjustable on the corresponding support bar for the purpose of adapting their position in order to place each one above a cavity of the plate during the operation of removing from the mould. The different bars 32 are movable on sidemembers 33 in the same way as it is with the previous implementation mode.

It is illustrated by FIG. 5 that in order to organize the gripping of the bread specimens corresponding to one-thirds of baguettes, with three bread specimens per cavity, it is provided the use of only three lines of prehensors. The used prehensors are carried by three of the bars 32, the other bars being deflected here and there relative to the area to be covered above the outstanding plate.

FIGS. 6A and 6B illustrate features of the invention concerning the constitution of the needles prehensors as they are used, preferably in the quality control installation illustrated for example. An individual needles prehensor is illustrated by FIG. 6A, in the appropriate bread-gripping position 8, while, by FIG. 6B, it is illustrated when the same bread specimen is released.

The bread specimen is effectively gripped by two curved needles that penetrate the inside of the bread by crossing each other. The two needles 74 and 75 are, to this effect, made articulated underneath a supporting plaque 76 and symmetrically curved towards each other. Each is free from its orientation around its articulation axis. On the other hand, it is guided at its other extremity in such a way that, once the prehensor is controlled to raise the plaque 76, the two needles get distanced from each other as they come out of the bread specimen 8. The guiding is realized in two openings 77 across a lower plaque 78; which is fixedly arranged with the bracket 72 which fixes the prehensor to the supporting bar. It is preferred that the openings 77 be limited by curved faces, like needles, to ensure a smooth guiding of the latter.

The prehensor automatically passes from one to the other of its functional positions as illustrated by FIGS. 6A and 6B. To this effect, the upper plaque 76 is mounted so that to slide freely on a guiding axis 79, which is incorporated with lower plaque 78. Between the two plaques, there is an expanding envelope 73, which contains an air cushion that authorizes their relative displacements. The pressure in the air cushion varies, and individually for each prehensor, according to the command of the driving system. It determines the height between the two plaques. In other words, each organ controlling needles exit or withdrawal functions like a pneumatic jack. When the prehensor system is lowered on the bread specimens that are on the operative plate, the driving system controls the displacement of the upper plaque 76 toward the lower plaque 78, and the two needles penetrate the bread located beneath. When the system goes up in reverse, the needles are disconnected from the bread and the plaque 76 is pneumatically solicited to move away from the plaque 78 during the rising.

The foregoing indicates how the invention can be advantageously implemented in quality control applications on bread specimens that are present in cavities of the plates, as cooking plates, how two successive visiometric inspections are realized, one of which using a digital images acquiring system which examines the bread specimens from above, and the other examining them from underneath while they are suspended from a transfer system which uses multiple individually controlled prehensors, how the flatness of the plates is checked using the same visiometric from above inspection, and, finally, how needle prehensors are advantageously used to seize bread specimens which, once extracted from the cooking place, are not hardened.

1. A process for controlling the quality of bakery products comprising:
submitting said products when present in their respective locations on a moulding plate to a first visiometric inspection step wherein they are inspected from above and digital image data are produced comprising at least location data indicative of their respective positions on said plate;
seizing individually said products out of said plate depending on said location data by prehensors arranged in a gripping matrix of prehensors; and
submitting said seized products to a second visiometric inspection step wherein they are inspected from underneath while they are hanging from said gripping matrix.

2. A process according to claim 1, comprising controlling said prehensors of said gripping matrix depending from location, presence, quality data obtained by data processing from digital image data acquired in said first and second visiometric inspection steps to release said seized products from said gripping matrix and lead each of them onto different circuits out depending on whether it conforms to predetermined quality specifications.

3. A process according to claim 2 wherein said plates are cooking plates that are driven out of an oven wherein said products moulded therein have been heated, wherein each said plate is checked for planeity at said first visiometric step after it has been emptied by seizing said products out of it, and wherein each said plate whose planeity is convenient for use is circulated back to said oven for moulding new products therein an heat them in said oven.

4. A quality control equipment in a bakery machine comprising:
first visiometric means for inspecting bakery products from above when present in their respective locations on a moulding plate and producing first digital image data comprising at least location data indicative of their respective positions on said plate;
transfer means comprising prehensors arranged in a gripping matrix of prehensors for individually seizing said products out of said plate; and
second visiometric means for inspecting said seized products from underneath while they are hanging from said matrix of prehensors and producing second digital image data at least indicative of their presence and/or quality for each of them.

5. A quality control equipment according to claim 4, wherein the first visiometric from above means comprise driving means of a digital camera scanning the upper side of the plate in operation.

6. A quality control equipment according to claim 5, wherein the digital camera used for the visiometric from above inspection is a camera for the acquisition of three dimensional images.

7. A quality control equipment according to claim 4, wherein the visiometric from underneath inspection is effected via a linear camera set in a fixed position, and wherein the gripping matrix is moving above the linear camera to transfer the seized products at a distance of the plate from which they have been extracted.

8. A quality control equipment according to claim 4, wherein the first visiometric from above means comprises driving means of a digital camera scanning the upper side of the plate in operation, wherein the visiometric from underneath inspection is effected via a linear camera set in a fixed position, and wherein the gripping matrix is moving above the linear camera to transfer the seized products away from the plate from which they have been extracted.

9. A quality control equipment according to claim 4, wherein said gripping matrix comprises suction prehensors, which are individually piloted relative to digital image data acquired in said visiometric inspection means.

10. A quality control equipment according to claim 4, wherein said gripping matrix comprises needles prehensors, which are individually piloted relative to digital image data acquired in said visiometric inspection means.

11. A quality control equipment according to claim 10, wherein said needles prehensors are pneumatically pivoted.

12. A quality control equipment according to claim 11, wherein the said piloting of the prehensors conducts a relative movement between a supporting plaque that is articulated by two needles facing each other and a plaque through which the needles are guided into respective openings.

13. A quality control equipment according to claim 4, wherein the visiometric from above means conducts a verification of the flatness of the plates via detection of the height of the flat areas between the cavities that are formed on said plates to define said products locations, for determining whether each plate can be used for another moulding operation, said visiometric from above means comprising a camera for the acquisition of three dimensional images.

14. A quality control equipment in a bakery machine comprising:
first visiometric means for inspecting bakery products from above when present in their respective locations on a moulding plate and producing first digital image data comprising at least location data indicative of their respective positions on said plate;
transfer means comprising prehensors arranged in a gripping matrix of prehensors for individually seizing said products out of said plate;
second visiometric means for inspecting said seized products from underneath while they are hanging from said matrix of prehensors and producing second digital image data at least indicative of their presence and/or quality for each of them; and
release control means for operating said prehensors to release said seized products from said matrix of prehensors and drive them to different circuits out depending upon location, presence and quality information obtained from said first and second digital image data acquired for each said products.

15. A bakery machine comprising:
first visiometric means for inspecting bakery products from above when present in their respective locations on a moulding plate and producing first digital image data comprising at least location data indicative of their respective positions on said plate;
transfer means comprising prehensors arranged in a gripping matrix of prehensors for individually seizing said products out of said plate;
second visiometric means for inspecting said seized products from underneath while they are hanging from said matrix of prehensors and producing second digital image data at least indicative of their presence and/or quality for each of them;
wherein said visiometric means are arranged at the end of an oven for cooking the said products present on said moulding plate that is here a cooking plate too; and
wherein means are provided for automatically ordering the transfer of each empty plate whose flatness has been checked as non-defective on a return conveyor towards the entry of the said cooking oven.
